(19) United States
(12) Patent Application Publication
(10) Pub. No.: US 2015/0283108 A1
(43) Pub. Date: Oct. 8, 2015

(54) SULFESTROL FOR TREATING CANCER

(71) Applicant: CHAMAELEO PHARMA BVBA, Diegem (BE)

(72) Inventors: Johannes Jan Platteeuw, Diegem (BE); Martijn De Bree, Almere (NL); Edwin Klumper, Amstelveen (NL)

(73) Assignee: CHAMAELEO PHARMA BVBA, Diegem (BE)

(21) Appl. No.: 14/435,733

(22) PCT Filed: Oct. 14, 2013
(86) PCT No.: PCT/EP2013/071438

§ 371 Date: Apr. 14, 2015

(30) Foreign Application Priority Data
Oct. 15, 2012 (EP) 12188502.4

(51) Int. Cl. A61K 31/255

(52) U.S. Cl. CPC A61K 31/255

(57) ABSTRACT

The invention relates to the use of Sulfestrol (diethylstilbestrol sulfate) in a method of curative or palliative treatment of cancer in a mammal. The inventors have unexpectedly found that Sulfestrol can suitably be employed as an effective drug in the treatment of cancer in mammals. The inventors have further discovered that Sulfestrol can be administered even in high dosages without giving rise to serious side effects. Besides the clinical use of Sulfestrol the present invention also relates to oral dosage units comprising Sulfestrol and to sterile liquids for intravenous administration that comprise Sulfestrol.

### TECHNICAL FIELD OF THE INVENTION

The present invention relates to the use of Sulfestrol (diethylstilbestrol sulfate) in curative or palliative treatment of cancer in a mammal, e.g. for use in the treatment of breast cancer or prostate cancer.

The invention also provides oral dosage units and sterile intravenous liquids comprising Sulfestrol.

### BACKGROUND OF THE INVENTION

Cancer is still among the major causes of death in the western world. This applies to both males and females. Due to ongoing research on new medicines and methods of treatment, life expectance of people suffering from different types of cancer has steadily increased over the years. Nevertheless, better medicines and enhanced methods of treatment are still needed.

Among the medicines that already have been used in the treatment of cancer is diethylstilbestrol (DES). DES is a synthetic non-steroidal estrogen that was first synthesized in 1938. It was designed to achieve castrate levels of testosterone. Testosterone drives prostate cancer growth and withdrawal of testosterone by surgical castration was the first hormone ablation therapy in prostate cancer treatment. DES was developed to achieve chemical castration by inhibiting testicular production of testosterone.

However, the role of oral administration of DES in the treatment of prostate cancer has been limited because of an association with thromboembolic toxicity. When estrogens like for example DES are given orally, they are subject to the intestinal and hepatic first-pass effect leading to high hormone concentrations in the liver promoting the synthesis of clotting proteins like fibrinogen.

Non-cancer related deaths, mostly cardiovascular in origin, were increased by 36% in patients suffering from prostate cancer receiving 5 mg of DES p.o. per day (Byar D P: Proceedings: The Veterans Administration Cooperative Urological Research Group's studies of cancer of the prostate. Cancer (1973) 32:1126-30). Other studies evaluating lower doses of DES reported similar efficacy towards testosterone suppression as obtained with the 5 mg dose and acceptable thromboembolic toxicity. This led to the adoption of 3 mg per day as the most commonly used DES oral dose for treating prostate cancer. However, the side effects remained a concern and DES was replaced as a first line therapy in prostate cancer by Leuprolide and by tamoxifen in breast cancer when studies were published that showed that the latter two therapies had similar efficacy but less side effects.

The concerns regarding the cardiovascular side effects have led to the development of DES-based formulations that are less prone to intestinal and hepatic first pass effect.

GB 732,286 describes the synthesis of DES diphosphate (fosfestrol). Fosfestrol was developed as a prodrug of DES to achieve safe inhibition of testosterone production without causing thromboembolic side effects caused by free DES. The phosphate groups were added to inactivate DES, thereby circumventing the intestinal and hepatic first pass effect and decreasing the circulating levels of free DES. Fosfestrol itself was considered to be inactive and it was known that prostate cancer cells have increased expression of prostate acid phosphatase (PAP). It was thought that PAP would remove the phosphate groups and release DES near its side of action.

Fosfestrol was introduced and marketed in the 1950′s under the name Honvan® and has been successfully applied in the treatment of prostate cancer for many years. However, fosfestrol was closely related to DES and when DES was replaced as first line therapy, fosfestrol was also side-lined.

The concerns regarding the cardiovascular side effects of DES even at low doses have also led to the development of formulations and routes of delivering DES that bypass the intestinal and hepatic first pass effect.

WO 2008/045461 A2 describes methods for treating prostate cancer comprising transdermally administering a therapeutically effective amount of DES or a pharmaceutically acceptable salt or complex thereof, to a subject suffering from prostate cancer. The transdermal DES may be used as a front line hormonal therapy or a second line hormonal therapy for treating prostate cancer. Transdermal administration may be accomplished via transdermal patches, lotions, creams, gels, pastes, sprays, ointments, eye drops, nose drops, ear drops, suppositories and/or similar transdermal administration techniques. DES may be administered in a dose of at least about 0.1 and more particularly about 5.0 mg/day. The maximum dosage is about 25 mg/day. The dose may be a single dose per day, it may be divided into at least two unit dosages for administration over a 24-hour period, or it may be a single continuous dose for a longer period of time, such as 3 days to 10 weeks or 1-10 weeks.

US 2011/0189288 A1 describes a way of circumventing the first pass effect by using a buccal pharmaceutical composition comprising a water soluble matrix comprising an effective amount of DES and an absorption enhancer having an HLB of 8 to 16. This US patent application also describes a method for treating prostate or breast cancer in a patient comprising administering the pharmaceutical composition as a water soluble matrix to the oral mucosal membranes of the mouth of a patient. In the treatment of both prostate cancer and breast cancer, from 0.1 mg to 15 mg per dose of DES is administered one to three times a day.

In summary, DES can be safely administered if it is given in a low oral dose of 3 mg daily, if it is given as a fosfestrol prodrug or if it is given via transdermal or buccal routes.

The in vitro effects of DES were studied to explore its carcinogenic and estrogenic characteristics. Brandes et al. (Receptor Status and Subsequent Sensitivity of Subclones of MCF-7 Human Breast Cancer Cells Surviving Exposure to Diethylstilbestrol, cancer research 43, 2831-2835, June 1983) investigated the estrogenic effects of DES on MCF-7 breast cancer cells to explore the potential use of DES in the treatment of breast cancer. They were the first to show that DES unexpectedly reduced cell growth in vitro.

Hartley-Asp et al. (Diethylstilbestrol induces metaphase arrest and inhibits microtubule assembly, Mutation Research, 143 (1985) 231-235) investigated the effects of DES on DU-145 prostate cancer cells in an attempt to elucidate the carcinogenic effects of DES. They showed cytotoxic effects of DES in the prostate cancer cells through inhibition of microtubule formation.

Schulz et al. (Evaluation of the Cytotoxic Activity of Diethylstilbestrol and Its Mono- and Diphosphate towards Prostatic Carcinoma Cells, Cancer Res. 48 (1988) 2867-2870) showed that DES concentrations ranging up to 100 ng/ml do not influence prostate cancer cell growth and that the minimal concentration of DES to induce cytotoxic effects in these cells is 1 μg/ml.

It is an object of the present invention to provide a method of treatment of cancer in a mammal that provides optimum efficacy by inducing androgen deprivation and/or cytotoxic effects with acceptable side effects.

### SUMMARY OF THE INVENTION

The present inventors have unexpectedly found that Sulfestrol (diethylstilbestrol sulfate) can suitably be employed as an effective drug in the treatment of cancer in mammals. Furthermore, the inventors have discovered that Sulfestrol can be administered even in high dosages without giving rise to serious side effects.

Without wishing to be bound by theory it is hypothesized that the toxic effects that have been observed in the past for orally administered DES (˜3 mg daily) are not so much caused by DES itself, but by oxidized DES metabolites. When DES is orally administered, it accumulates in the liver where it is oxidized. Therefore, bypassing liver metabolism of DES is key to reduce thromboembolic side effects.

Without wishing to be bound by theory it is hypothesized that Sulfestrol is a prodrug of DES and that Sulfestrol is less susceptible to intestinal and hepatic first pass effect than DES. Consequently, following administration of Sulfestrol, a reservoir of Sulfestrol is created which is either at least partly converted in the liver to DES by sulfatase (arylsulfatase C) or is converted to DES at the place of the carcinoma by sulfatase enzymes expressed by the tumor cells. The liberated DES from the liver sulfatases enters the circulation and DES accumulation in the liver is minimized.

It is believed to be important that Sulfestrol is administered in a relatively high amount so as to achieve a cytotoxic effect.

Sulfate esters of DES have been described in the prior art.

Giacomelli et al. (TERAPIA ASSOCIATA ORMONICA-CHIRURGICA-RADIOLOGICA DEL CANCRO INOPERABILE DELLA MAMMELLA, Tumori, It. Vol. 21 (1947), 338-345) describe a trial in which diethylestilbestrol-disulphate was administered intravenously to female humans with inoperable breast cancer. The authors report a favorable impact of this treatment in combination with radiotherapy and surgery.

Cavallini (UN NUOVO DERIVATO STILBENICO INIETTABILE PER VIA ENDOVENOSA, Bollettion della societa Italiana di biologia sperimetnale, Napoli, IT, vol. (1947),214-216) concludes that diethylstilbestrol-disulfate has estrogenic activity, is water soluble, intravenously injectable and well tolerated in mice

Cavallini et al. (TERAPIA DEL CANCRO E SOSTANZE A BASSÄ ATTIVITA ESTROGENA, Il Farmaco, Elsevier France, Editions scientifiques et medicales, IT, vol. 3, (1948), 300-303) investigated the estrogenic activity of disulphuric esters (sodium salts) of 4,4′-dioxy-benzoin, 4-4′-dioxydesoxybenzoin, 4-4′-dioxy-ethyldesoxybenzoin, 3,4-di (p.oxyphenyl)n-hesan3-ol by injecting ovariectomized female rats with a olive oil containing these substances.

Bishop et al. (Stilbestrol sulphate, oestrone, and equilin: further observations on the potency and clinical assessment of oestrogens, The Lancet, 1951, Apr. 13; 818-820) studied the estrogenic effect of a range of sulfated estrogens. The study was started as previous results had shown that preparations containing estrone sulfate had more estrogenic activity than the parent compound estrone. The authors wondered whether the sulfate form of stilbestrol would also prove to be more estrogenic. In this study stilbestrol sulfate was orally administered in daily doses of 2 mg. The study showed that stilbestrol sulfate had 44% of the estrogenic activity of stilbestrol.

Demol et al. (Influence des steroids sexuels sur la croissance du carcinoma mammaire T.M. 2290 A.A.L. cheze les souris males de la race O₂₀ A.A.L″, ANNALES D′ENDOCRINOLOGIE, MASSON, PARIS, FR, vol. 16, (1955) 932-937) describe a study in which tumors were induced into male mice (castrated or non-castrated) by subcutaneous injection of a suspension of meshed tumor tissue. Subsequently, the mice were exposed to disodium disulfate of diethylstilbestrol using two different methods of administration: subcutaneous injection (12×0.16 mg) or by addition to the drinking water (0.04, 0.1 and 0.2 mg/day).

Saruta et al. (The Mechanism of Estrogen Hypertension, JAPANESE CIRCULATION JOURNAL, vol. 36, no. 6, (1972), 611-616) discuss the effect of estrogens in oral-contraception on hypertension in rats. The article describes the intraperitoneal injection of 2 mg stilbestrol disulfate in combination with 0.2 mg estriol intramuscularly to male rats.

Curtis and his coworkers used 35-S radioactive labeled DES sulfates in their studies to elucidate the behavior of arylsulfatase C and arylsulfate esters in vivo. More specifically, Gregory et al. (The fate of the sulphate esters of diethylstilboestrol in the rat, Biochem J. 1971 December; 125(3): 77-78) and Bradford et al. (Metabolic Fates of Diethylstilboestrol Sulphates in the Rat, Biochem J. 1977; 164: 423-430) used DES[³⁵S] monosulfate and DES[³⁵S]disulfate amongst others as model compounds. Mature free-ranging male and female rats received injections of DES[³⁵S]disulfate. In addition, DES[³⁵S]monosulfate was administered to anaesthetized rats with bile-duct and ureter cannulae. The authors found that both compounds are billiary excreted without further metabolic modifications, making them ideal model compounds for their metabolic studies towards understanding the activity of arylsulfatase C

As arylsulfatase C was specifically found in the liver the authors identified the liver as the major site of metabolism of both DES disulfate and DES monosulfate.

Besides the clinical use of Sulfestrol the present invention also relates to oral dosage units comprising Sulfestrol and to sterile liquids for intravenous administration that comprise Sulfestrol.

### DETAILED DESCRIPTION OF THE INVENTION

A first aspect of the invention concerns Sulfestrol (diethylstilbestrol sulfate) for use in a method of curative or palliative treatment of cancer in a mammal.

The term ‘Sulfestrol’ as used herein, unless indicated otherwise, refers to a diethylstilbestrol moiety of which at least one of the hydroxyl groups is sulfated. Hence, Sulfestrol as used herein includes Mono-Sulfestrol (diethylstilbestrol monosulfate or 4-(4-(4-oxidophenyl)hex-3-en-3-yl)phenyl sulfate), Di-Sulfestrol (diethylstilbestrol disulfate or 4,4′-(hex-3-ene-3,4-diyl)bis(4,1-phenylene) disulfate) and mixtures thereof. The remaining hydroxylgroup of Mono-Sulfestrol may be esterified with e.g. a phosphate group. The term ‘Sulfestrol’ also encompasses pharmaceutically acceptable salts of Sulfestrol.

The term ‘pharmaceutically acceptable salt’, as used herein, means those salts of compounds of the invention that are safe and effective for use in mammals and that possess the desired biological activity. Descriptions of counter ions for pharmaceutically acceptable salts of pharmaceutical compounds can be found in P. Heinrich Stahl, Camille G. Wermuth (Eds.), Handbook of Pharmaceutical Salts, Properties, Selection and Use, Wiley VCH (2002).

The diethylstilbestrol moiety in the Sulfestrol of the present invention may be in the trans-form or the cis-form. Naturally, also mixtures of the trans- and cis-form may be employed.

The term ‘cancer’ as used herein refers to a malignant neoplasm involving unregulated cell growth. In cancer, cells divide and grow uncontrollably, forming malignant tumors, and invade nearby parts of the body.

The term ‘curative treatment’ as used herein refers to a treatment that aims to cure a disease or to improve symptoms associated with a disease.

The term ‘palliative treatment’ as used herein refers to a treatment or therapy that does not aim at curing a disease but rather at providing relief

The term ‘oral’ as used herein, unless indicated otherwise, is synonymous to ‘per oral’.

The term ‘dosage’ as used herein refers to the amount of a pharmaceutically active substance that is administered to a mammal. Hence, the term ‘dosage’ does not include any carrier or other pharmaceutically acceptable excipient that is part of a ‘dosage unit’ to be administered.

In this document and in its claims, the verb ‘to comprise’ and its conjugations are used in their non-limiting sense to mean that items following the word are included, without excluding items not specifically mentioned. In addition, reference to an element by the indefinite article ‘a’ or ‘an’ does not exclude the possibility that more than one of the element is present, unless the context clearly requires that there be one and only one of the elements. The indefinite article ‘a’ or ‘an’ thus usually means ‘at least one’.

Hormone-dependent cancers refer to those types of cancer that grow faster in the presence of particular hormones. This type of cancer is usually treated with hormone therapy. Hormone therapy involves blocking in vivo production or action of these hormones. Therefore, hormone therapy actually is anti-hormone therapy. Cancer of the prostate, breast, cervix, endometrium and ovaries can be hormone-dependent cancers and may be treated in accordance with the present method.

In the case of hormone-dependent prostate cancer, androgen ablation therapy (e.g. orchiectomy, treatment with LHRH analogs or LHRH antagonists) is used as first line treatment to decrease the production of androgens, particularly testosterone, in order to stop or limit the growth of prostate cancer. Androgens are key drivers of prostate tumor growth. The androgen ablation therapies reduce the plasma levels of androgen, thereby reducing the growth potential of the prostate tumor. The androgen ablation therapies are successful for a certain period of time, however all prostate tumors eventually become resistant to this treatment approach. After failure of the androgen ablation therapy, secondary hormone treatments with anti-androgens are used to slow the growth of the prostate tumor.

Breast cancer is commonly classified on the basis of its receptor status. Breast cancer cells may or may not have many different types of receptors, the three most important in the present classification being: estrogen receptor (ER), progesterone receptor (PR), and HER2/neu. The ER/PR positive breast cancers have the most favorable clinical outcome as they are very responsive to anti hormone treatments. If one of these receptors is not expressed by the tumor, hormone therapies are less effective. If none of the two receptors are expressed, the tumor is insensitive to hormone treatments and the cancer is called ‘hormone-independent’. Besides the ER and PR, breast cancers can also express the protein HER2/neu. Expression of HER2/neu is correlated with a more aggressive tumor and a poorer clinical outcome compared to HER2/neu negative tumors. If all three markers described above are not expressed the breast cancer is called ‘triple negative’.

After exposure for a certain time to hormone therapy most of the cancer types mentioned before obtain the ability to grow without hormones and become called ‘hormone-independent’. Once these cancers become hormone-independent, treatment usually is switched to chemotherapy. Hormone-independent prostate cancer is also called hormone-refractory or castration-resistant prostate cancer. These terms are used interchangeably in the following and are considered to have the meaning of ‘castration-resistant prostate cancer’. Nowadays, the term ‘castration resistant’ has replaced ‘hormone refractory’ because while these prostate cancers are no longer responsive to castration treatment (reduction of available androgen/testosterone), they still show some reliance upon hormones for androgen receptor activation.

The present invention encompasses the treatment of hormone-dependent as well as hormone-independent cancers. The present method is particularly suited for treatment of hormone-independent cancers, especially for treatment of hormone-independent cancers that have developed after treatment of hormone dependent cancers with hormone therapy. The present method is particularly suited for treatment of hormone independent cancers that have developed after treatment of hormone dependent cancers with anti-estrogen, aromatase inhibitor, anti-androgen or an inhibitor of 17a hydroxylase/C17,20 lyase (CYP17A1).

The present method of treatment is advantageously applied to treat a breast cancer that does not respond to treatment with anti-estrogen, aromatase inhibitor or an inhibitor of 17α hydroxylase/C17,20 lyase (CYP17A1).

The present method may also advantageously be applied to treat a prostate cancer that does not respond to treatment with anti-androgen or an inhibitor of 17α hydroxylase/C17,20 lyase (CYP17A1), especially a prostate cancer that does not respond to treatment with an inhibitor of 17α hydroxylase/C17,20 lyase (CYP17A1), more particularly to treatment with Abiraterone.

In a preferred embodiment of the invention, the cancer treated is prostate cancer, particularly castration-resistant prostate cancer.

In another preferred embodiment, the cancer treated by the present method is breast cancer, especially ER/PR negative breast cancer. In still another advantageous embodiment the method of treatment refers to the treatment of triple-negative breast cancer.

In a preferred embodiment the Sulfestrol is Mono-Sulfestrol. In a further preferred embodiment, the Mono-Sulfestrol is not DES glucuronide sulfate. Preferably, at least one of the hydroxyl groups in the diethylstilbestrol moiety of the Mono-Sulfestrol is not esterified.

In another preferred embodiment the Sulfestrol is Di-Sulfestrol.

As explained herein before, Sulfestrol in the context of the present invention also encompasses pharmaceutically acceptable salts of Sulfestrol. Pharmaceutically acceptable salts include those formed from cations of alkali metals such as sodium, lithium, potassium, and earth alkali metals such as calcium and magnesium.

In a preferred embodiment the Sulfestrol is an alkali metal salt, notably a sodium and/or a potassium salt. More preferably, the Sulfestrol is in the potassium salt form.

The present method of treatment may be used to treat several kinds of mammals, e.g. humans, horses, cattle etc. The present method is particularly suited for the treatment of humans.

The Sulfestrol dosage may vary depending upon the specific conditions and patients undergoing treatment. The therapeutically effective dosage of the compound can be provided as repeated doses within a prolonged treatment regimen that will yield clinically significant results.

The actual dosage of the compound will vary according to factors such as the disease indication and particular status of the subject such as for example, age, size, fitness, extent of symptoms, susceptibility factors and the like, and other factors such as time and route of administration, other drugs or treatments being administered concurrently. Dosage regimens can be adjusted to provide an optimum therapeutic response.

Typically, the present method comprises administering Sulfestrol in a daily dosage of at least 0.001 mmol, more preferably of at least 0.01 mmol, even more preferably at least 0.03 mmol, yet more preferably of at least 0.1 mmol and most preferably at least 0.2 mmol. The daily administered dose of Sulfestrol preferably does not exceed 15 mmol, more preferably it does not exceed 5 mmol, most preferably it does not exceed 2 mmol.

Expressed differently, it is preferred to administer Sulfestrol in a daily amount of at least 0.013 μmol per kg of bodyweight, more preferably of at least 0.13 μmol per kg of bodyweight, even more preferably of at least 0.4 μmol per kg of bodyweight and most preferably of at least 1.3 μmol per kg of bodyweight. The daily administered amount of Sulfestrol preferably does not exceed 200 μmol per kg of bodyweight, more preferably it does not exceed 80 μmol per kg of bodyweight most preferably it does not exceed 26 μmol per kg of bodyweight.

The duration of the present method of treatment typically exceeds 7 days. More particularly, the present method has duration of at least 14 days, especially of at least 28 days.

In accordance with the present invention the Sulfestrol is preferably administered orally, intravenously, topically or transmucosally. Naturally, also combinations of these modes of administration may be employed. According to a particularly preferred embodiment the Sulfestrol is administered orally and/or intravenously. Most preferably, the Sulfestrol is administered orally.

In accordance with a particularly preferred embodiment of the invention the Sulfestrol is administered orally in a daily dosage of at least 0.001 mmol, more preferably of at least 0.01 mmol, even more preferably of at least 0.03 mmol, yet more preferably of at least 0.1 mmol, and most preferably of at least 0.2 mmol. The orally administered daily dosage preferably does not exceed 15 mmol, more preferably it does not exceed 5 mmol and most preferably it does not exceed 2 mmol

It is further preferred to orally administer Sulfestrol in a daily amount of at least 0.013 μmol per kg of bodyweight, more preferably of at least 0.13 μmol per kg of body weight, even more preferably of at least 0.4 μmol per kg of body weight and most preferably of at least 1.3 μmol per kg of bodyweight. The orally administered daily amount of Sulfestrol typically does not exceed 200 μmol per kg of body weight, more preferably it does not exceed 80 μmol per kg of body weight and most preferably it does not exceed 26 μmol per kg of body weight.

The aforementioned daily dosage may be administered once daily of it may be administered in the form of two or more separate doses at more or less regular intervals. According to a particularly preferred embodiment, the present method of treatment comprises orally administering at least two doses of each at least 0.001 mmol Sulfestrol per day, more preferably it comprises orally administering at least 3 doses of at least 0.001 mmol Sulfestrol per day. Preferably, the latter doses each contain at least 0.01 mmol, more preferably at least 0.03 mmol and most preferably at least 0.05 mmol Sulfestrol.

Another preferred route of administration is intravenous administration, i.e. infusion of a Sulfestrol containing pharmaceutical formulation directly into the veins. Compared with other routes of administration, the intravenous route is the fastest way to deliver medications throughout the body.

Typically, the present method comprises intravenously administering Sulfestrol in a daily dosage of at least 0.001 mmol, more preferably of at least 0.01 mmol, even more preferably of at least 0.03 mmol, yet more preferably of at least 0.1 mmol and most preferably at least 0.2 mmol. The intravenously administered daily amount of Sulfestrol typically does not exceed 15 mmol, more preferably it does not exceed 5 mmol, most preferably it does not exceed 2 mmol.

Expressed differently, it is preferred to intravenously administer Sulfestrol in a daily amount of at least 0.013 μmol per kg of bodyweight, more preferably of at least 0.13 μmol per kg of body weight, even more preferably of at least 0.4 μmol per kg of body weight and most preferably of at least 1.3 μmol per kg of bodyweight. Preferably, the daily administered amount of Sulfestrol does not exceed 200 μmol per kg of body weight, more preferably it does not exceed 80 μmol per kg of body weight and most preferably it does not exceed 26 μmol per kg of body weight.

Typically, intravenous administration of the Sulfestrol comprises administration of an intravenous dose of at least 0.001 mmol, more preferably of at least 0.01 mmol, even more preferably of at least 0.03 mmol, yet more preferably of at least 0.1 mmol and most preferably of at least 0.2 mmol. Typically, the intravenous dose does not exceed 15 mmol, more preferably it does not exceed 2 mmol. The present method of treatment preferably comprises administration of at least one intravenous dose of Sulfestrol per day.

Another aspect of the invention relates to an oral dosage unit comprising at least 0.001 mmol, preferably at least 0.01 mmol, more preferably at least 0.03 mmol and most preferably at least 0.1 mmol of Sulfestrol other than DES mono[³⁵S]sulfate or DES di[³⁵S]sulfate. Typically, the oral dosage unit comprises not more than 15 mmol, more preferably not more than 5 mmol and most preferably not more than 2 mmol Sulfestrol other than DES mono[³⁵S]sulfate or DES di[³⁵S]sulfate.

The terms DES mono[³⁵S]sulfate and DES di[³⁵S]sulfate as used herein refer to Mono-Sulfestrol and Di-Sulfestrol which have been isotopically labeled with ³⁵5-atoms. Preferably, the various possible S-isotopes are present in the oral dosage unit in their natural (relative) abundances.

The oral dosage unit of the present invention can be advantageously applied in the methods of treatment of curative or palliative treatment of cancer as defined herein before.

The oral dosage units is preferably selected from the group consisting of tablets, granulates, capsules and powders and liquids. Even more preferably, the oral dosage unit is a tablet or capsule.

The oral dosage units typically have a weight of between 0.1 and 2 g, more preferably of 0.15 and 1.0 g and most preferably of 0.2-0.5 g.

The oral dosage units typically comprise between 10 and 95 wt. % of one or more pharmaceutically acceptable excipients selected from coloring agents, flavoring or taste masking agents, diluents, binders, lubricants, disintegrants, stabilizers, surfactants, glidants, plasticizers, preservatives and sweeteners.

The excipient is advantageously chosen from the group consisting of lactose, anhydrous lactose, crospovidone, croscarmellose sodium, sodium starch glycolate, hydroxypropyl cellulose, polacrilin potassium, pregelatinized starch, microcrystalline cellulose and combinations thereof. In a preferred embodiment the oral dosage units comprise up to 5 wt. %, preferably 2-4 wt. % of disintegrants.

The dosage unit of the present invention may suitably take the shape of a compressed tablet. Such a tablet may suitably comprise two or more layers of different composition, for example a core comprising Sulfestrol as defined herein before encased in a coating.

The dosage units of the present inventions are conveniently produced in a tabletting machine. In order to enable easy removal of the tablets from the moulds, the dosage unit typically contains between 0.5 and 4 wt. % of a lubricant or gliding agent. Preferably, the lubricant or gliding agent is selected from the group consisting of talc, sodium stearyl fumarate, magnesium stearate, calcium stearate, hydrogenated castor oil, hydrogenated soybean oil, polyethylene glycol, starches, anhydrous colloidal silica and combinations thereof.

Another aspect of the invention relates to sterile liquid for intravenous administration, said liquid containing at least 0.1 micromol/ml, preferably at least 0.5 micromol/ml, even more preferably at least 3 micromol/ml and most preferably at least 10 micromol/ml, of Sulfestrol other than DES mono[³⁵S]sulfate or DES di[³⁵S]sulfate. Typically, the sterile liquid comprises not more than 1,200 micromol/ml, more preferably not more than 700 micromol/ml and most preferably not more than 400 micromol/ml Sulfestrol other than DES mono[³⁵S]sulfate or DES di[³⁵S]sulfate.

Preferably, the various possible S-isotopes are present in the DES-disulfate contained in the liquid for intravenous administration in their natural (relative) abundances.

The sterile liquids for intravenous administration of the invention can be advantageously applied in the methods of treatment of curative or palliative treatment of cancer as defined herein before.

Sterile liquids for intravenous administration are prepared by dissolving Sulfestrol and other pharmaceutically acceptable excipients in liquid inert carriers. A suitable inert liquid carrier is Water for Injection.

In addition, the presence of a tonicity agent, e.g. sodium chloride in an amount of about of 1-8 mg/ml, to adjust the tonicity to the same value of human blood is required. In general, the osmolarity of the formulation resembles that of human blood. Typically, the osmolarity of the sterile liquid is in the range of 270 to 310 mOsm/kg, more preferably in the range of 275 to 300 mOsm/kg, and most preferably in the range of 280-290 mOsm/kg.

The pH of the sterile liquid preferably lies in the range of 4-8, more preferably in the range of 5-7.

Non-limiting examples of other suitable excipients for intravenous formulations are solvents such as ethanol, glycerol and propylene glycol, stabilizers like EDTA (ethylene diamine tetraacetic acid) and citric acid, antimicrobial preservatives like benzyl alcohol, methyl paraben and propyl paraben, buffering agents like citric acid/sodium citrate, acetic acid/sodium acetate and phosphoric acid/potassium dihydrogen phosphate, and tonicity modifiers such as sodium chloride, mannitol and dextrose.

Persons skilled in the art are aware of the limitations that are applicable to formulations for intravenous administration. The formulation should not contain excipients that would cause an adverse reaction when entered into the blood. Furthermore, the formulation should allow for the active ingredient to remain soluble once entered into the blood. This list of limitations is not exhaustive. It is within the skills of the artisan to select appropriate excipients that meet these requirements.

The following examples are meant to further illustrate the invention and some of its preferred embodiments without intending to limit its scope.

### EXAMPLES

### Example 1

Objective of the experiments described in this example was the synthesis of 10-25 g of both Mono-Sulfestrol and Di-Sulfestrol.

The synthesis of these compounds on a milligram scale was published in Biochem. J. (1977), 164, 423-430. The synthesis of the disulfate of DES, as described in this publication, starts with mixing ClSO₃H, CS₂ and N,N-dimethyl aniline at −5° C.. after which diethylstilbestrol is added at room temperature.

Test reactions using these conditions afforded mixtures of mono- and disulfate. Using ClSO₃H and DMAP in pyridine to which diethylstilbestrol was added also resulted in mixtures of the mono- and disulfate of DES.

However, when the reaction was performed in N,N-dimethylformamide, using an excess of pyridine-SO₃ complex as reactant, almost complete conversion of DES to its disulfate was observed. Remaining starting material was removed by washing the reaction mixture with Et20 (diethyl ether). Subsequent crystallization from water, followed by crystallization of the product from aqueous KOH solution removed any remaining mono-sulfate and pyridine residues, and 27 g of DES disulfate was obtained as the corresponding di-potassium salt.

### Synthesis of Di-Sulfestrol

The synthesis of Di-Sulfestrol was performed as follows:

Diethylstilbestrol (30 g, 112 mmol) was dissolved in N,N-Dimethylformamide (dry, 100 ml). Sulfur trioxide pyridine complex (71.2 g, 447 mmol) was added to the solution and the mixture was stirred at ambient temperature for 3 hrs.

A white solid precipitated and a sample was filtered off. LCMS (PEVA39-013-3) performed on this sample indicated a mixture of Mono-Sulfestrol and Di-Sulfestro land also some starting material.

The remainder of the precipitate was filtered off and was subsequently washed on the filter with ethanol (2×200 ml). LCMS indicated a mixture of Mono-Sulfestrol (6%) and Di-Sulfestrol (94%). The solid was recrystallized from 500 ml 25% KOH solution to afford crude Di-Sulfestrol as white crystals. The crystals were filtered off and dried in vacuo to remove any residual solvent.

LCMS indicated a product having a purity higher than >99% and correct mass (m/z=213 (428-2H⁺/2)).

¹H-NMR was in agreement with structure. All aromatic signals appeared as one big singlet. ¹³C-NMR (D₂O) showed 7 peaks, all in agreement with the structure of Di-Sulfestrol.

Synthesis of the monosulfate could be achieved using 0.4-0.5 equivalents of pyridine-SO₃ in DMF. This resulted in only minor amounts of the disulfate as a side-product. Again, washing the reaction mixture with Et20 removed any unreacted starting material. Crystallization of the mixture from H₂O afforded the monosulfate in good purity.

However, when the product was treated with aqueous KOH solution to remove the pyridine residue, a side product was formed. Attempts to fully remove this unknown side product by crystallization or washing turned out to be unsuccessful and a purity of Mono-Sulfestrol as a dipotassium salt to just above 90% was obtained.

### Synthesis of Mono-Sulfestrol

The synthesis of Mono-Sulfestrol was performed as follows:

Diethylstilbestrol (40 g, 149 mmol) was dissolved in N,N-Dimethylformamide (dry, 50 ml). Sulfur trioxide pyridine complex (9.49 g, 59.6 mmol) was added to the solution and the mixture was stirred at ambient temperature for 3 hrs. A solid precipitated and was filtered off. LCMS performed on this sample indicated a 30% conversion to Mono-Sulfestrol and the presence of very little Di-Sulfestrol.

The solvent was evaporated in vacuo from the remainder of the precipitate affording crude Mono-Sulfestrol as a white solid. The crude product was partially redissolved in hot water (80-90 ° C.) and filtered hot. The filtrate was cooled to ambient temperature and was filtered of LCMS was performed on a sample of the retentate and indicated a 85:15 mixture of Mono-Sulfestrol and DES starting material.

The remainder of the retentate was stirred in ethanol and filtered off. The filtrate was then stirred in 10% KOH solution for 10 minutes and the solid was filtered off The crude product was once more recrystallized from ethanol in 2 batches, yielding respectively 210 mg and 1.75 gram of Mono-Sulfestrol as a dipotassium salt. LCMS analysis indicated that the 210 mg batch had a purity of about 92% and that still some ethanol was present. The 1.75 gram batch had a LCMS purity of about 91%

### Example 2

The in vitro direct cytotoxicity of DES, Mono-Sulfestrol and Di-Sulfestrol in hormone-dependent (LNCaP) and hormone-independent (DU-145) prostate cancer cell lines was tested.

Cells were maintained in vitro in RPMI 1640 containing 10% (v/v) heat inactivated fetal bovine serum (FBS) and 2 mM L-glutamine (growth media) at 37° C. in 5% CO₂ and humidified conditions. Cells were harvested, washed, re-suspended into growth medium and counted. The cells were re-suspended into assay media (RPMI 1640+1% (v/v) heat inactivated FBS+ and 2 mM L-glutamine) at 0.5×10⁵ cells/ml for DU-145 cells and 1×10⁵ for LNCaP cells, and plated into 96-well assay plates (Corning, black-wall plates) using 50 μl/well aliquots.

Plates were incubated overnight at 37° C. in 5% humidified CO₂ prior to addition of the compounds. Three compounds (DES, Mono-Sulfestrol and Di-Sulfestrol) were dissolved in 100% DMSO at stock concentration of 60 mM.

Stocks of all compounds were then serially diluted. Final concentrations to which cells were exposed were 300, 150, 75, 37.5, 18.75, 9.4, 4.7, 2.3, 1.2 and 0.6 μM. Positive control was Taxotere. Taxotere was diluted in 100% DSMO to give a stock concentration of 1 mM. Stock was serially diluted. Final concentrations to which cells were exposed were 1000, 333.3, 111.1, 37.0, 12.3, 4.1, 1.4, 0.5, and 0.2 nM.

Plates were incubated for 72 hrs at 37° C. in 5% humidified CO₂ after addition of the compounds. Viability of the cells was assessed with the Cell titer blue® (Promega) assay. 10 μl of Cell titer Blue reagents was added to each test/blank well. Plates were incubated for 3 hrs at 37° C. in 5% humidified CO₂ prior to analysis. Fluorescence was measured with a Flex II station plate reader. Excitation wavelength was 570 nm, emission wave length was 600 nm, cut off was 590 nm. Raw data was processed by GraphPad Prism to calculate mean, standard deviation and IC₅₀ values.

Results on the viability of the cell lines exposed to DES, Mono-Sulfestrol, Di-Sulfestrol and Taxotere are shown in Table 1.

TABLE 1

IC₅₀ value (μM)

| | LNCaP | DU-145 |
|---|---|---|
| DES | 27 | 62 |
| Mono-Sulfestrol | Not active | Not active |
| Di-Sulfestrol | Not active | Not active |
| Taxotere (control) | 0.002 | 0.004 |

### Conclusion

These results show that DES is cytotoxic in vitro in LNCaP and DU-145 prostate cancer cells. Mono-Sulfestrol and Di-Sulfestrol are both inactive.

### Example 3

The in vitro direct cytotoxicity of DES, Mono-Sulfestrol and Di-Sulfestrol in hormone-dependent (MCF-7) and hormone-independent (MDA-MB321) triple negative breast cancer cell lines was tested.

Cells were maintained in vitro in RPMI 1640 containing 10% (v/v) heat inactivated FBS and 2 mM L-glutamine (growth media) at 37° C. in 5% CO₂ and humidified conditions. Cells were harvested, washed, resuspended into growth medium and counted. The cells were re-suspended into assay media (RPMI 1640+1% (v/v) heat inactivated FBS+ and 2 mM L-glutamine) at 0.5-1×105 cells/ml (dependent upon cell type) and plated into 96-well assay plates (Corning, black-wall plates) in 50 ul/well aliquots.

Plates were incubated overnight at 37° C. in 5% humidified CO₂ prior to addition of the compounds. Three compounds (DES, Mono-Sulfestrol and Di-Sulfestrol) were dissolved in 100% DMSO at stock concentration of 60 mM. Stocks of all compounds were then serially diluted. Final concentrations to which cells were exposed were 300, 150, 75, 37.5, 18.75, 9.4, 4.7, 2.3, 1.2, 0.6 μM. Positive control was Cisplatin. Cisplatin was diluted in 1% FBS media to give a stock concentration of 1 mM. Cisplatin stock was then serially diluted. Final concentrations to which cells were exposed were 100, 50, 25, 12.5, 6.3, 3.1, 1.6, 0.8, 0.4, 0.2 μM.

Plates were incubated for 72 hrs at 37° C.. in 5% humidified CO₂ after addition of the compounds. Viability of the cells was assessed with the Cell titer blue® (Promega) assay. 10 μl of Cell titer Blue™ reagents was added to each test/blank well. Plates were incubated 3 h at 37° C. in 5% humidified CO₂ prior to analysis. Fluorescence was measured with a Flex II station plate reader. Excitation wavelength was 570 nm, emission wave length was 600 nm, cut off was 590 nm. Raw data was processed by GraphPad Prism to calculate mean, standard deviation and IC₅₀ values. Results on the viability of the cell lines exposed to DES, Mono-Sulfestrol, Di-Sulfestrol and Cisplatin are shown in Table 2.

TABLE 2

IC₅₀ value (μM)

| | MCF7 | MDA-MB-231 |
|---|---|---|
| DES | 3.9 | 6.7 |
| Mono-Sulfestrol | 85 | 185 |
| Di-Sulfestrol | Not active | Not active |
| Cisplatin (control) | 2.8 | 13 |

### Conclusions

DES and Mono-Sulfestrol are cytotoxic in vitro in MCF7 and MDA-MB231 breast cancer cell lines. DES is the most active compound, followed by Mono-Sulfestrol. Di-Sulfestrol is inactive in these cells.

### Example 4

The platelet-aggregation-inducing effects of DES, Mono-Sulfestrol and Di-Sulfestrol in platelet-rich plasma (PRP) were investigated.

Blood necessary to generate PRP was obtained from 4 healthy human volunteers. 100 ml of fresh venous blood from each donor was isolated directly into tubes containing 1/10th volume of the anti-coagulant Tri-Sodium Citrate (3.2% w/v). The blood and anti-coagulant were immediately mixed and the final concentration of Tri-Sodium Citrate was 0.0106 M.

PRP was prepared by centrifugation of anti-coagulated blood at 180 g for 10 minutes at room temperature. The cloudy yellow supernatant containing the platelets was removed and placed into a clean polypropylene tube. Platelet-poor plasma (PPP) was prepared by centrifuging the remaining sample at 1,200 g for 20 minutes at room temperature. The PRP was adjusted to a suitable platelet count and absorbance at 630 nm by addition of PPP.

All three compounds were dissolved in 100% DMSO to give a stock concentration of 60 mM. Stocks were serially diluted and final concentrations to which the platelets were exposed were 150, 75, 37.5, 18.75, 9.4, 4.7 and 2.3 μM. The final concentrations tested were 100, 33, 11.1, 3.7, 1.2, 0.4 and 0.133 nM. 100 nM gamma thrombin (Cambridge bioscience) was used as control for platelet aggregation. 90 μl aliquots of PRP were added to 96 wells plates. A baseline absorbance reading was taken at 630 nm. 5 μl of the compounds and gamma thrombin control was added to the appropriate PRP.

The plates were incubated at room temperature for 7 minutes on a plate shaker with continuous shaking A second A630 nm reading was made after the 7 minutes incubation to measure any platelet aggregation induced by the compounds. Raw data was processed by GraphPad Prism to calculate mean and standard deviation. The results so obtained are shown in Table 3.

TABLE 3

| | Absorbance (630 nm) | | | |
|---|---|---|---|---|
| Concentration | | Mono- | | |
| [μM] | DES | Sulfestrol | Di-Sulfestrol | Control |
| 2.3 | 0.264 ± 0.030 | 0.274 ± 0.034 | 0.260 ± 0.030 | |
| 4.7 | 0.266 ± 0.031 | 0.277 ± 0.029 | 0.253 ± 0.024 | |
| 9.4 | 0.264 ± 0.030 | 0.272 ± 0.029 | 0.266 ± 0.039 | |
| 18.75 | 0.264 ± 0.030 | 0.275 ± 0.029 | 0.254 ± 0.025 | |
| 37.5 | 0.264 ± 0.030 | 0.278 ± 0.030 | 0.260 ± 0.027 | |
| 75 | 0.268 ± 0.032 | 0.281 ± 0.031 | 0.264 ± 0.031 | |
| 150 | 0.263 ± 0.033 | 0.283 ± 0.036 | 0.266 ± 0.034 | |
| Control | | | | 0.030 ± 0.005 |

### Conclusion

All compounds show a much higher absorbance compared to the thrombin control. Concentration of Thrombin was set at 100 ng/ml as this was the optimal concentration to induce platelet aggregation. As high absorbance correlates to low aggregation, these results show that none of the compounds can directly induce platelet aggregation.

### Example 5

In vitro metabolism pathways of DES, Mono-Sulfestrol and Di-Sulfestrol in human liver microsomes were investigated.

Special emphasis was put on desulfatation by steroid sulfatase (STS) and oxidation by cytochrome P (CYP) metabolism. Liver microsomes (Celsis in vitro Technologies) were used at a concentration of 0.5 mg/ml in a volume of 600 μl phosphate buffer pH 7.4. Estrone-3-sulfate was used as positive control.

In order to distinguish STS metabolism from CYP metabolism, four different incubations with different situations were tested.

1. nothing added; STS is active and CYP enzymes are inactive.
2. addition of NADPH; STS and CYP enzymes are active.
3. addition of STS inhibitor 667-Coumate; both STS and CYP enzymes are inactive.
4. addition of 667-Coumate and NADPH; STS is inactive and CYP enzymes are active.

5 μM of each compound (test and control compound) was added to the liver microsomes and the mixture was incubated at 37° C. for 5, 10, 20, 30, 50, 90 and 120 minutes. After incubation, aliquots of 45 μl were taken which were subsequently quenched with 150 μl ice cold methanol. Aliquots were incubated ≧1 h at −20° C. for protein precipitation. After precipitation, samples were centrifuged and remaining supernatant was used for analysis with LC-MS/MS. Half life of the compounds, speed of metabolism or intrinsic clearance (Clint), and contribution to metabolism of CYP and STS metabolism were calculated based on the data obtained at each time point.

The results so obtained are shown in Tables 4a, 4b, 4c and 4d.

TABLE 4a

| | | Clint | Contribution |
|---|---|---|---|
| | | (fil/min/mg | to metabolism |
| DES disappearance | Half-life (min) | protein) | (%) |
| DES + 667-Coumate | n.d. | n.d. | n.d. |
| DES + NADPH | 18 | 76.0 | 100 |
| DES | 194 | 7 | 9 |
| DES + NADPH + | 14 | 100 | 131 |
| 667-Coumate | | | |

TABLE 4b

| | | Clint | Contribution |
|---|---|---|---|
| Mono-Sulfestrol | Half-life | (fil/min/mg | to metabolism |
| disappearance | (min) | protein) | (%) |
| Mono-Sulfestrol + | n.d. | n.d. | n.d. |
| 667-Coumate | | | |
| Mono-Sulfestrol + NADPH | 12 | 114.3 | 100 |
| Mono-Sulfestrol | 63 | 22 | 19 |
| Mono-Sulfestrol + | 27 | 51 | 44 |
| NADPH + 667-Coumate | | | |

TABLE 4c

| | | | Contribution |
|---|---|---|---|
| | | Clint | to |
| | Half-life | (fil/min/mg | metabolism |
| Di-Sulfestrol disappearance | (min) | protein) | (%) |
| Di-Sulfestrol + 667-Coumate | 697 | 2.0 | 5 |
| Di-Sulfestrol + NADPH | 38 | 36.2 | 100 |
| Di-Sulfestrol | 45 | 31 | 85 |
| Di-Sulfestrol + NADPH + 667- | n.d. | n.d. | n.d. |
| Coumate | | | |

TABLE 4d

| | | Clint | Contribution |
|---|---|---|---|
| Estrone-3-S | | (fil/min/mg | to metabolism |
| disappearance | Half-life (min) | protein) | (%) |
| E-3-S + 667-Coumate | n.d. | n.d. | n.d. |
| E-3-S + NADPH | 12 | 115.7 | 100 |
| E-3-S | 27 | 51 | 44 |
| E-3-S + NADPH + | 62 | 22 | 19 |
| 667-Coumate | | | |

### Conclusion

These results show that DES is only processed by CYP metabolism, giving rise to oxidative metabolites. Mono-Sulfestrol is metabolized by both STS and CYP enzymes. Metabolism of Mono-Sulfestrol by STS enzymes results in the production of DES, which is then further metabolized by CYP enzymes. Mono-Sulfestrol is also directly metabolized by CYP enzymes, resulting in oxidative metabolites of Mono-Sulfestrol. Di-Sulfestrol is only metabolized by STS enzymes. Di-Sulfestrol is almost completely converted to Mono-Sulfestrol, which is then further metabolized by STS and CYP enzymes.

### Example 6

The metabolites of Mono-Sulfestrol and Di-Sulfestrol in plasma were measured in male Sprague Dawley rats. Metabolites were identified after oral and intravenous administration of the compounds.

Each group consisted of three rats, giving a total of 12 animals. Compounds were dosed at 10 mg/kg for both oral and intravenous administration. A 0.9% saline solution was used as vehicle for oral and intravenous administration.

Prior to intravenous administration, the solution was filtered through a 0.22 μM filter. Oral dosing was performed using a oral gavage. Tail vein injection was used for intravenous administration.

At 0.5, 1, 2, 4, 6 and 8 hrs post administration, 0.25 ml blood was drawn from the animals and immediately transferred to prechilled microcentrifuge tubes containing 4 μl of K2-EDTA to prevent coagulation. Plasma was obtained by centrifugation of the plasma and stored at −70° C. until analysis. Plasma drawn from each individual rat was pooled according to the following schedule:

| Time point | Sample volume (μl) |
|---|---|
| t₀ | 10.0 |
| t₁ | 30.0 |
| t₃ | 60.0 |
| t₄ | 80.0 |
| t₅ | 80.0 |
| t₆ | 40.0 |
| Total | 300 |

The pooled samples were analyzed using LC-MS/MS and the relative percentage of each metabolite in the pooled sample was measured on the basis of the mass spectrometric peak areas of the parent and its metabolites in the plasma. The results so obtained are shown in Tables 5a and 5b.

TABLE 5a

| | | Retention | Relative | |
|---|---|---|---|---|
| | [M − H]⁻ | Time | Abundance | |
| Metabolite | m/z | (min) | (%) | Metabolic Pathway |
| M1 | 363.1 | 7.76 | IV: 0.74 | Oxidation, |
| | | | PO: 0.00 | (P + O) |
| M2 | 297.1 | 7.82 | IV: 0.00 | Desulfation |
| | | | PO: 0.51 | and oxidation |
| | | | | and methylation, |
| | | | | (P − SO₃ + O + CH₂) |
| M3 | 377.1 | 9.34 | IV: 7.22 | Oxidation |
| | | | PO: 2.98 | and methylation, |
| | | | | (P + O + CH₂) |
| P (Mono- | 347.1 | 9.48 | IV: 86.99 | |
| Sulfestrol) | | | PO: 91.91 | |
| M4 | 363.1 | 9.60 | IV: 0.00 | Oxidation, |
| | | | PO: 0.82 | (P + O) |
| M5 | 377.1 | 9.65 | IV: 3.19 | Oxidation |
| | | | PO: 2.97 | and methylation, |
| | | | | (P + O + CH₂) |
| M6 | 377.1 | 10.80 | IV: 1.86 | Oxidation |
| | | | PO: 0.81 | and methylation, |
| | | | | (P + O + CH₂) |

P = Parent;
M = Metabolite;
IV = Intravenous Administration;
PO = Oral administration

TABLE 5b

| | | Retention | Relative | |
|---|---|---|---|---|
| | [M − H]⁻ | Time | Abundance | |
| Metabolite | m/z | (min) | (%) | Metabolic Pathway |
| M1 | 523.1 | 9.02 | IV: 1.11 | Desulfation and |
| | | | PO: 0.00 | glucuronidation, |
| | | | | (P − SO₃ + C₆H₈O₆) |
| P (Di- | 427.1 | 13.17 | IV: 5.09 | |
| Sulfestrol) | | | PO: 8.41 | |
| M2 | 443.1 | 13.54 | IV: 1.18 | Oxidation, |
| | | | PO: 1.37 | (P + O) |
| M3 | 457.1 | 14.35 | IV: 1.81 | Oxidation |
| | | | PO: 1.19 | and methylation, |
| | | | | (P + O + CH₂) |
| M4 | 523.1 | 14.61 | IV: 0.35 | Desulfation and |
| | | | PO: 0.00 | glucuronidation, |
| | | | | (P − SO₃ + C₆H₈O₆) |
| M5 | 347.1 | 16.35 | IV: 84.22 | Desulfation, |
| | | | PO: 86.77 | (P − SO₃) |
| M6 | 377.1 | 16.40 | IV: 3.29 | Desulfation |
| | | | PO: 2.26 | and oxidation |
| | | | | and methylation, |
| | | | | (P − SO₃ + O + CH₂) |
| M7 | 377.1 | 16.82 | IV: 2.96 | Desulfation |
| | | | PO: 0.00 | and oxidation |
| | | | | and methylation, |
| | | | | (P − SO₃ + O + CH₂) |

P = Parent;
M = Metabolite;
IV = Intravenous Administration;
PO = Oral Administration

### Conclusions

These results show that circulating Mono-Sulfestrol undergoes minimal metabolic processing. In addition, oral administration of Mono-Sulfestrol is favorable over intravenous dosing in terms of achieving the highest Mono-Sulfestrol plasma levels. There is some oxidative metabolism of Mono-Sulfestrol detected, however the levels of metabolites are low. Di-Sulfestrol is mostly converted into Mono-Sulfestrol, the latter is the most abundant circulating metabolite of Di-Sulfestrol. Some oxidative metabolism and glucuronide conjugation is detected, however at low levels.

### Example 7

The concentration of Mono-Sulfestrol in the pooled samples described in Example 6 was quantified by LC-UV quantification.

A calibration standard solution was prepared as 10,000 ng/mL by adding an appropriate amount of Mono-Sulfestrol into 20% acetonitrile/0.1% formic acid in water. The peak area of the standard solution and the sample were measured with the wavelength set at 200 to 360 nm. The concentration levels of Mono-Sulfestrol were estimated using peak area (Area) of the standard and unknown samples and concentration of the standard (Conc_{std}) by the following equation:

Area_{unknown}/Area_{std}=Conc_{unknown}/Conc_{std}

Conc_{unknown}=Conc _{std}(Area_{unknown}/Area_{std})

The results are shown in table 6

TABLE 6

| Sample | Mono-Sulfestrol (ng/ml) | Mono-Sulfestrol (μM) |
|---|---|---|
| Mono-Sulfestrol | 630.0 | 1.5 |
| intravenous | | |
| Mono-Sulfestrol oral | 1150.6 | 2.7 |
| Di-Sulfestrol | 735.9 | 1.7 |
| intravenous | | |
| Di-Sulfestrol oral | 261.3 | 0.6 |

### Example 8

The estrogenic activity of DES and Di-Sulfestrol was measured in male Sprague-Dawley rats. Estrogenic activity was determined by measuring body weight and plasma cholesterol levels.

18 Sprague-Dawley rats were randomly divided into three groups (vehicle, DES and Di-Sulfestrol, n=6). Each animal was dosed three times a day with vehicle or test compound (1^{st} dose 7:30-8:00, 2^{nd} dose 12:30-13:00, 3^{rd} dose 17:00-17:30) for 14 consecutive days. DES was dosed at 0.5mg/kg per dosing time, total dose was 1.5 mg/kg per day. Di-Sulfestrol was dosed at 1 mg/kg per dosing time, total dose was 3 mg/kg per day.

Body weight was measured on days 0, 2, 4, 6, 8, 10, 12 and 14 between 13:30 and 13:00.

After 14 days treatment, blood was collected from the animals via cardiac puncture 4 hrs past final dosing to analyze plasma cholesterol levels. The blood was immediately transferred to a tube containing 0.5M EDTA anticoagulant. It was mixed gently and centrifuged at 1000×g at 4° C. for 15 min. Plasma was aspirated from the centrifuged blood samples, quick frozen on dry ice and stored at −80° C. for later use.

Plasma cholesterol levels were determined using a commercial ELISA kit obtained from NovaTeinBio (BG-RAT11262). Standard protocol accompanied by the kit was used without any adjustments.

The results of the body weight and plasma cholesterol measurements are shown in Tables 7a and 7b.

TABLE 7a

| | Mean bodyweight | | | | | | | |
|---|---|---|---|---|---|---|---|---|
| Days | 0 | 2 | 4 | 6 | 8 | 10 | 12 | 14 |
| DES | 382 ± | 379 ± | 361 ± | 350 ± | 342 ± | 341 ± | 337 ± | 339 ± |
| | 7.8 | 9.3 | 8.6 | 8.0 | 8.0 | 7.8 | 8.4 | 9.80 |
| Di-Sulfestrol | 387 ± | 381 ± | 368 ± | 357 ± | 348 ± | 344 ± | 341 ± | 333 ± |
| | 6.9 | 8.3 | 9.5 | 9.2 | 8.5 | 7.2 | 7.5 | 6.67 |
| Vehicle | 379 ± | 391 ± | 399 ± | 407 ± | 423 ± | 429 ± | 441 ± | 452 ± |
| | 5.9 | 7.4 | 7.8 | 6.6 | 8.4 | 9.8 | 9.8 | 8.47 |

TABLE 7B

| | Cholesterol |
|---|---|
| | (nmol/L) |
| DES | 14.9 ± 0.724 |
| Di-Sulfestrol | 12.1 ± 0.799 |
| Vehicle | 222 ± 32.2 |

### Conclusions

Both DES and Di-Sulfestrol show estrogenic activity by lowering the body weight and cholesterol levels in rats. Unlike DES, however, Di-Sulfestrol was not cytotoxic in vitro in breast and prostate cancer cell lines, as shown by the in vitro cytotoxicity data (see Examples 2 and 3).

This data indicates that Di-Sulfestrol is desulfated in the rats, thereby releasing DES and the corresponding estrogenic activity. Likewise, in vivo desulfation of Di-Sulfestrol will release the cytotoxic activity of DES.

### Example 9

An experiment is performed to determine the in vivo efficacy of Mono-Sulfestrol.

A validated in vivo breast cancer model is used to determine the effects of Mono-Sulfestrol on tumor growth and progression. To this end tumors are grown in a group of animals, preferably in mice

Once the tumors are established oral treatment with Mono-Sulfestrol and Di-Sulfestrol is started. During the treatment the size of the tumor is monitored.

The results of the experiment indicate that Mono-Sulfestrol, when administered in adequate doses, can be used to treat breast cancer.

### Example 10

An experiment is performed to determine the in vivo efficacy of Di-Sulfestrol.

A validated in vivo prostate cancer model is used to determine the effects of Di-Sulfestrol on tumor growth and progression. To this end tumors are grown in a group of animals.

Once the tumors are established oral treatment with Di-Sulfestrol is started. During the treatment the size of the tumor is monitored. Overall survival of the animals is used to measure the efficacy of the treatment.

The results of the experiment indicate that Di-Sulfestrol, when administered in adequate doses, can be used to treat prostate cancer.

### Example 11

A 1 kg batch of 250 mg tablets containing 50 mg of Mono-Sulfestrol was prepared as follows:

200 gram of Mono-Sulfestrol, 160 grams of silicified microcrystalline cellulose, 570 grams of lactose monohydrate and 50 grams of croscarmellose sodium were each passed separately over an 710 micron sieve. The sieved materials were placed in a tumble blender and mixed for 20 minutes. In the mean time 20 grams of magnesium stearate was passed through a 500 micron sieve. The material was added to the tumble blender and mixing was continued for another 5 minutes.

Tablets with a weight of 250 mg were prepared on a Korsch EKO using 9.5 mm diameter round punches.

### Example 12

Capsules containing 50 mg of Di-Sulfestrol were prepared by weighing 50 grams of Di-Sulfestrol, 40 grams of microcrystalline cellulose, 140 grams of lactose monohydrate and 4 grams of colloidal silica . After passing all separate materials through a 710 micron sieve. The materials were transferred to a V-blender and blended for 15 minutes.

Hard gelatin capsules size 2 were prepared with an average fill weight of 234 mg.

### Example 13

The effects of mono-Sulfestrol and di-Sulfestrol on tumor progression were determined in an orthotopic spontaneous metastasis breast model using bioluminescent MDA-MB-231 Dlux injected into the mammary fat pad (MFP) of female MF-1 nude mice.

Female MF-1 nude mice (HsdOla:MF1-Foxnlnu, Harlan, UK) were divided into 3 groups of each 8 mice (n=8):

1) mono-Sulfestrol
2) di-Sulfestrol
3) vehicle negative control

The mice were 5-6 weeks old at the start of the study and were maintained in sterile isolators within a barriered unit illuminated by fluorescent lights set to give a 12 hour light-dark cycle (on 07.00, off 19.00). The room was air-conditioned to maintain an air temperature range of 23±2° C. Sterile irradiated 2019 rodent diet (Harlan Teklad UK, product code Q219DJ1R2) and autoclaved water were offered ad libitum.

The MDA-MB-231 Dlux cells were maintained in vitro in RPMI containing 2 mM L-Glut and (Life Technologies, UK) and 10% (v/v) heat-inactivated foetal bovine serum (Sigma, Poole, UK) at 37° C. in 5% CO₂ and humidified conditions. Cells were treated with Zeocin once a week at 0.4 mg/mL.

On the day of initiation cells were harvested from semi-confluent monolayers with 0.05% Trypsin-EDTA, washed twice in the culture medium and counted. Cells with viability of >90% were re-suspended, for in vivo administration, in 1:1 PBS:Matrigel at 2×10⁶ cells/0.1 mL. Prior to injection, the cell suspension was homogenized and 0.1 mL of cell suspension was drawn into a 1 mL syringe. The cell suspension was then injected orthotopically into the lower left-side 2nd mammary fat pad of each mouse under anaesthesia (Medetomidine/Ketamine).

On day 7 after initiation and every six days thereafter, the mice were injected (s.c.) with 150 mg/kg D-Luciferin. 10 minutes following administration of D-Luciferin mice were anaesthetised and 15 minutes after administration they were placed into an imaging chamber (Spectrum CT) and imaged for luminescence (ventral view; whole body and shielding of the primary tumor). Images were captured and processed by Living Image 4.3.1 software (Caliper LS, US.).

On day 7 after initiation mice were assigned to groups such that there was a uniform mean bioluminescent signal between the groups.

Immediately after assignment, treatment was started:

Group 1: received 60 mg/kg mono-Sulfestrol in WFI (water for injection) orally once a day
Group 2: received 80 mg/kg di-Sulfestrol in WFI orally once a day.
Group 3: received PEG/water for injection in a 50/50 mixture orally once a day

The results obtained are shown in the Table 8. This table gives the total flux (photons/second) measured±the Standard Deviation in 10⁹ units determined by whole body bioluminescence measurements as described above.

TABLE 8

| | Day 7 (flux * 10⁹) | Day 13: (flux * 10⁹) | Day 19 (flux * 10⁹) |
|---|---|---|---|
| Group 1 | 1.72 | 5.89 | 9.22 |
| Group 2 | 1.73 | 8.84 | 16.2 |
| Group 3 | 1.72 | 13.4 | 23.1 |

This data shows that both mono-Sulfestrol and di-Sulfestrol have a reducing effect on the signal intensity, indicating that both compounds have a tumor progression inhibiting effect when orally administered to a mouse in an adequate dose.

1-21. (canceled)

22. A method of curative or palliative treatment of breast cancer or prostate cancer in a mammal, comprising administering to the mammal a mono-sulfestrol (diethylstilbestrol-monosulfate) selected from the group consisting of 4-(4-(4-oxidophenyl)hex-3-en-3-yl)phenyl sulfate, pharmaceutically acceptable salts and esters thereof, other than 4,4′-(hex-3-ene-3,4-diyl)bis(4,1-phenylene) disulfate.

23. The method according to claim 22, wherein the cancer is a hormone independent cancer.

24. The method according to claim 23, wherein the hormone independent cancer has become hormone independent after treatment of a hormone dependent cancer with hormone therapy.

25. The method according to claim 24, wherein the hormone independent cancer has become hormone independent after treatment of a hormone dependent cancer with anti-estrogen, aromatase inhibitor, anti-androgen or an inhibitor of 17α hydroxylase/C17,20 lyase (CYP17A1).

26. The method according to claim 22, wherein the administration is oral and/or intravenous.

27. The method according to claim 26, wherein the method comprises daily oral administration of at least 0.03 mmol of mono-sulfestrol.

28. The method according to claim 27, wherein the method comprises daily oral administration of at least 0.2 mmol mono-sulfestrol.

29. The method according to claim 26, wherein the method comprises daily intravenous administration of at least 0.03 mmol mono-sulfestrol.

30. The method according to claim 29, wherein the method comprises daily intravenous administration of at least 0.2 mmol mono-sulfestrol.

31. An oral dosage unit comprising at least 0.001 mmol of mono-sulfestrol, wherein the mono-sulfestrol is not diethylstilbestrol mono[³⁵S]sulfate.

32. The oral dosage unit according to claim 31, comprising at least 0.03 mmol of the mono-sulfestrol.

33. The oral dosage unit according to claim 31, in the form of a tablet or capsule.

34. A sterile aqueous liquid for intravenous administration, comprising at least 0.1 μmol/m1 of mono-sulfestrol other than diethylstilbestrol mono [³⁵S]sulfate.

35. The sterile aqueous liquid according to claim 34, having an osmolality of 270-310 mOsm/l.
